(19) United States
(12) Patent Application Publication
(10) Pub. No.: US 2025/0242068 A1
(43) Pub. Date: Jul. 31, 2025

(54) RADIANT CATALYTIC IONIZATION DETOXIFICATION SYSTEM AND APPLICATION THEREOF AND RADIANT CATALYTIC IONIZATION DETOXIFICATION METHOD

(71) Applicant: QINGDAO AGRICULTURAL UNIVERSITY, Qingdao (CN)

(72) Inventors: Qingli YANG, Qingdao (CN); Yongchao MA, Qingdao (CN); Ping LIU, Qingdao (CN); Xiudan HOU, Qingdao (CN); Yinglian ZHU, Qingdao (CN); Jian JU, Qingdao (CN); Fuguo XING, Qingdao (CN); Yutao WANG, Qingdao (CN); Zijian WU, Qingdao (CN)

(21) Appl. No.: 19/080,899

(22) Filed: Mar. 16, 2025

Related U.S. Application Data
(63) Continuation of application No. PCT/CN2023/095433, filed on May 22, 2023.

(30) Foreign Application Priority Data
Apr. 21, 2023 (CN) 202310432102.X

(51) Int. Cl. A61L 2/08; A23B 2/00; A23B 2/50; A23B 2/53; A23B 20/30; A61L 2/10; A61L 2/14; B01J 23/888; B01J 35/39; B01J 37/04; B01J 37/06; B01J 37/08; B01J 37/34; C01G 49/00; C02F 1/32; C02F 1/72

(52) U.S. Cl. CPC A61L 2/088; A23B 2/001; A23B 2/503; A23B 2/53; A23B 20/30; A61L 2/10; A61L 2/14; B01J 23/888; B01J 35/39; B01J 37/04; B01J 37/06; B01J 37/08; B01J 37/343; C01G 49/0018; C02F 1/325; C02F 1/725; A61L 2202/11; A61L 2202/122; A61L 2202/123; C01P 2002/72; C01P 2002/82; C01P 2002/84; C01P 2004/03; C01P 2004/04; C02F 2305/10

(57) ABSTRACT

Disclosed is a radiant catalytic ionization detoxification system including a gas-liquid mixer configured to gas-liquid mix air carrying radiant catalytic ionized Reactive Oxygen Species with liquid material to be detoxified, and a reaction tube configured to circulate the mixed gas-liquid mixture. The detoxification system of the present disclosure is applicable to water sterilization or aflatoxin removal in edible oils. The radiant catalytic ionization chamber in the system can provide the system with air containing Reactive Oxygen Species, wherein mesh panels coated with photocatalytic materials are configured inside the chamber body, which configuration not only increases the photocatalytic material content per unit volume, but also expands the light-exposed surface area due to uniform distribution of the photocatalytic materials on the mesh panels. The system uses a U-shaped tube as the reaction tube, and the length of the reaction tube can be freely designed according to the practical detoxification requirements.

This application is a Continuation Application of PCT/CN2023/095433, filed on May 22, 2023, which claims priority to Chinese Patent Application No. CN 202310432102.X, filed on Apr. 21, 2023, which is incorporated by reference for all purposes as if fully set forth herein.

### TECHNICAL FIELD

The present disclosure relates to technology field of radiant catalytic ionization device, and in particular to, radiant catalytic ionization detoxification system and application thereof and radiant catalytic ionization detoxification method.

### BACKGROUND

Radiant catalytic ionized Reactive Oxygen Species are obtained by Radiant Catalytic Ionization (RCI). The Radiant Catalytic Ionization (RCI) is an emerging biocidal detoxification technology that can be used without restriction in human, animal and plant environments as it does not produce potentially hazardous by-products such as ozone, formaldehyde or carbon monoxide. The core of this technology is the photocatalytic effect, i.e., the absorption of light energy with the help of photocatalytic materials generates electron (e⁻) and hole (h⁺) pairs, which are converted into reactive oxygen species (ROS) with strong oxidising power and participate in the redox reaction of organic matter. When this technology is used for detoxification and sterilization in prior art, it is carried out under experiment laboratory conditions, and the liquid needs to be in direct contact with the photocatalytic material during the experiments, which poses certain food safety risks. Meanwhile, there is no suitable device to complete treatments of detoxification and sterilization of a large amount of liquid materials under production conditions.

Photocatalytic materials are crucial for radiant catalytic ionization technology, different photocatalytic materials have different efficiency in generating Reactive Oxygen Species, and at present, there are fewer types of photocatalytic materials available, and the more conventional ones are TiO₂, ZnO, and so on.

### SUMMARY

Regarding to the problems existing in the prior art, the present disclosure proposes a radiant catalytic ionization detoxification system, includes a gas-liquid mixer configured to gas-liquid mix air carrying radiant catalytic ionized Reactive Oxygen Species with liquid material to be detoxified, and a reaction tube configured to circulate the mixed gas-liquid mixture.

Based on the above solution, the reaction tube includes a plurality of U-shaped tubes, and a connecting tube configured to connect the U-shaped tubes; an air supplement tube is detachably connected at a distal end of a straight tube section of the U-shaped tubes, the air supplement tube being configured to introduce air carrying radiant catalytic ionized Reactive Oxygen Species into the U-shaped tubes; a plurality of air supplement holes are disposed on a wall of the air supplement tube.

Based on the above solution, a plurality of fins are disposed on the air supplement tube.

Based on the above solution, the air carrying radiant catalytic ionized Reactive Oxygen Species are provided by a radiant catalytic ionization chamber; the radiant catalytic ionization chamber includes a chamber body, a plurality of mesh panels disposed in the chamber body and a lamp tube configured to provide a radiant light source; a photocatalytic material which produces Reactive Oxygen Species upon light irradiation is coated on the mesh panels.

Based on the above solution, a fixing frame is further disposed in the chamber body, the fixing frame being configured to fix the mesh panels and the lamp tube; a fixing hole configured to fix the lamp tube, and a fixing rod configured to fix the mesh panels are disposed on the fixing frame; a mounting hole configured to be passed through by the lamp tube, and a fixing hole configured to be passed through by the fixing rod are disposed on the mesh panels.

Based on the above solution, the photocatalytic material is FeWO₄-rGO composite material.

The present disclosure further provides a method for preparing FeWO₄-rGO composite material. Specifically, the method consists of the following steps:

### (1) Preparation of FeWO₄

Firstly dissolving 5 mmol FeCl₃·6H₂O and 5 mmol Na₂WO₄·2H₂O respectively into 25 ml distilled water. Then, adding ascorbic acid into FeCl3 solution during continuous stirring for complete dissolution. Next, slowly adding Na₂WO₄ solution into the above mixture. after further stirring for 30 min, adding the mixture into 100 mL autoclave (the autoclave was only used as a reaction vessel), maintaining the mixture at 150-200° C. for at least 9 h. Then naturally cooling down the autoclave to room temperature. Collecting a resulting precipitate by centrifugation, and washing several times with distilled water and anhydrous ethanol, and drying in air at 80° C. for 6 h.
wherein: the molar ratio of Fe³⁺: ascorbic acid is: 5:0.2-5:1;

### (2) Preparation of FeWO₄-rGO Composite Material

Firstly adding 2 g FeWO₄ into 300 mL ethanol and ultrasonicating (ultrasonic condition 300 W) for 30 min. After adding 2 mL APTES into FeWO₄ suspension, heating the mixture at 70° C. for 4 h. Subsequently collecting the powder, washing with ethanol for several times, then drying it at 80° C. for overnight. Afterwards, adding 1 g APTES-modified FeWO₄ into 60 mL distilled water, and ultrasonic treating (ultrasonic condition 300 W) for 15 min, and then adding 0.05 g rGO. After stirring for 60 min, transferring a resulting suspension into an autoclave, and maintaining it at 180° C. for 12 h. Collecting a resulting product, washing with water, and drying at 80° C. overnight, to obtain FeWO₄-rGO.

The above radiant catalytic ionization detoxification system can be used for water sterilization or aflatoxin removal in edible oils.

The present disclosure further provides a radiant catalytic ionization detoxification method, applied to the above-described system, includes a step of generating Reactive Oxygen Species according to radiant catalytic ionization, a step of gas-liquid mixing air carrying radiant catalytic ionized Reactive Oxygen Species with liquid materials to be detoxified, and a step of detoxifying bacteria or aflatoxin in liquid materials by Reactive Oxygen Species.

The detoxification system of the present disclosure can be used for water sterilization or aflatoxin removal in edible oils, The radiant catalytic ionization chamber in the system can provide the system with air containing Reactive Oxygen Species wherein mesh panels coated with photocatalytic materials are disposed inside the chamber body, not only increases the photocatalytic material content per unit volume, but also expands the light-exposed surface area due to uniform distribution of the photocatalytic materials on the mesh panels. The system employs a U-shaped tubes as the reaction tube, and the length of the reaction tube can be freely designed according to the practical detoxification requirements, and the installation is convenient and fast, In order to enhance the detoxification efficiency of the system, the system of the present disclosure uses an air supplement tube to continuously replenish new air carrying radiant catalytic ionized Reactive Oxygen Species. Meanwhile, regarding to the photocatalytic material used in the present disclosure, i. e., the FeWO₄-rGO composite material, the material is prepared in a simple method, and the method uses ascorbic acid not only as a reducing agent but also as a structure-directing agent to synthesize the FeWO₄ product. And the surface charge modification provides a reasonable method for constructing a FeWO₄-rGO nanocomposite photocatalyst with sufficient interfacial contact according to electrostatic self-assembly. The FeWO₄-rGO composite material prepared by the method of the present disclosure exhibits a strong photocatalytic performance.

### BRIEF DESCRIPTION OF DRAWINGS

FIG. 1 illustrates a principle schematic diagram of a system described in embodiment 1;

FIG. 2 illustrates a structure schematic diagram of a radiant catalytic ionization chamber in the detoxification system described in the present disclosure;

FIG. 3 illustrates a structure schematic diagram of mesh panels in the detoxification system described in the present disclosure;

FIG. 4 illustrates a structure schematic diagram of a reaction tube in the detoxification system described in the present disclosure;

FIG. 5 illustrates a structure schematic diagram and a partially enlarged schematic diagram of an air supplement tube in the detoxification system described in the present disclosure;

FIG. 6 illustrates a structure schematic diagram of a fixing frame in the detoxification system described in the present disclosure;

FIG. 7 illustrates a schematic diagram of an XRD pattern of the sample in embodiment 2, wherein, (a) snowflake-like FeWO₄, (b) FeWO₄-rGO-U, and (c) FeWO₄-rGO;

FIG. 8 illustrates a schematic diagram of a Raman spectrum of the sample in embodiment 2, wherein, (a) GO, (b) FeWO₄, and (c) FeWO₄-rGO.

FIG. 9 illustrates a schematic diagram of a UV-vis diffuse reflectance spectrum of samples in embodiment 2, wherein, (a) FeWO₄, (b) FeWO₄-rGO-U, and (c) FeWO₄-rGO;

FIG. 10 illustrates a schematic diagram of N2 adsorption and desorption isotherms of the sample in embodiment 2; wherein, (a) snowflake-like FeWO₄, (b) FeWO₄-rGO-U, and (c) FeWO₄-rGO;

FIG. 11 illustrates scanning electron microscopy (SEM) and transmission electron microscopy images of the samples in embodiment 2; (a, b) snowflake-like FeWO₄, (c) SEM images of FeWO₄-rGO composite material, (d) (e) HRTEM, and (f) corresponding SAED of FeWO₄-rGO composite materials;

FIG. 12 illustrates a schematic reaction mechanism diagram of the photocatalytic redox reaction on the FeWO₄-rGO composite photocatalyst under visible light irradiation;

FIG. 13 illustrates SEM images of FeWO₄ generated under different temperature conditions; wherein, (a) 120° C., (b) 150° C., (c, d) 200° C.;

FIG. 14 illustrates SEM patterns and XRD patterns of FeWO₄ generated under different time conditions; (a) 0 h, (b) 2 h, (c) 6 h, (d) 9 h, and (e) 12 h. (f) XRD patterns of the prepared samples after hydrothermal treatment at 180° C. for different reaction times;

FIG. 15 illustrates a schematic diagram of oxidation process of L-ascorbic acid;

FIG. 16 illustrates a schematic diagram of forming FeWO₄ with snowflake-like structure;

FIG. 17 illustrates SEM images of FeWO₄ generated under different ascorbic acid dosage conditions, wherein: molar ratios of Fe³⁺: ascorbic acid are different, wherein, (a, b) 5:0.2, (c, d) 5:0.3, and (e, f) 5:1;

FIG. 18 illustrates SEM images of FeWO4 prepared from different raw materials, wherein, (a, b) Fe (NO₃)₃ was used instead of FeCl3; (c, d) 5 mmol FeCl₃·6H₂O, 5 mmol Na₂WO₄·2H₂O, 0.5 mmol ascorbic acid and 0.1 mmol of cetyltrimethylammonium bromide.

### DETAILED DESCRIPTION OF THE EMBODIMENTS

Terms used in the present disclosure, unless otherwise indicated, generally have the meanings commonly understood by those of ordinary skill in the art.

Hereinafter, the present disclosure is described in further detail in connection with specific embodiments and with reference to data. The following embodiments are only for the purpose of exemplifying the present disclosure, and are not intended to limit the scope of the present disclosure in any way.

### Embodiment 1

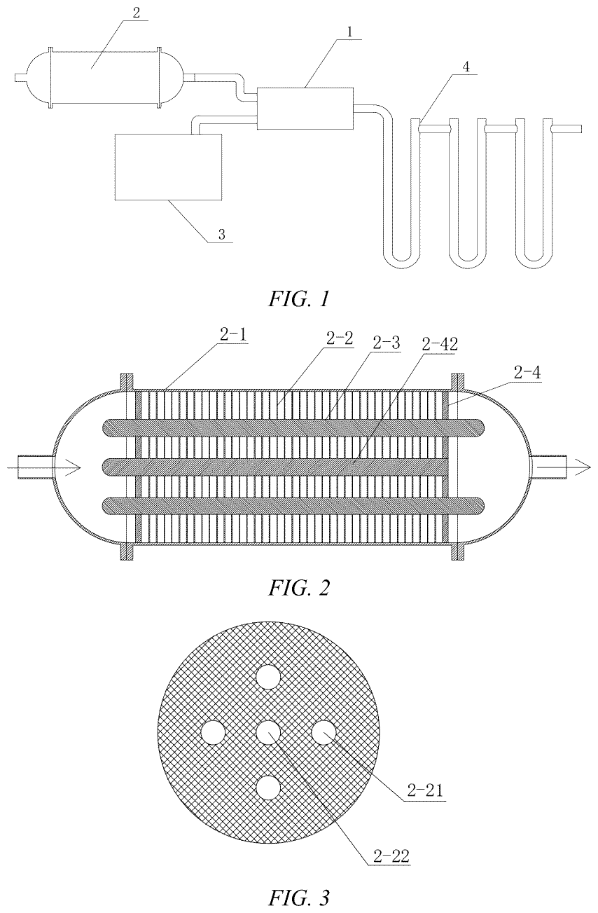

As shown in FIG. 1, the present disclosure provides a radiant catalytic ionization detoxification system, wherein the system includes a gas-liquid mixer 1 configured to gas-liquid mix air carrying radiant catalytic ionized Reactive Oxygen Species with liquid material to be detoxified, and a reaction tube 4 configured to circulate the mixed gas-liquid mixture.

The radiant catalytic ionized Reactive Oxygen Species are obtained by means of Radiant Catalytic Ionization (RCI) technology. The radiant catalytic ionization (RadiantCatalyticIonization, RCI) is an emerging bactericidal detoxification technology that can be used without restriction in human, animal and plant environments as it does not produce potentially dangerous by-products such as ozone, formaldehyde or carbon monoxide. The core of this technology is the photocatalytic effect, i.e., generating electron (e⁻) and hole (h⁺) pairs by absorbing light energy with the help of photocatalytic materials, and converting them into Reactive Oxygen Species (ROS) with strong oxidising capacity and participate in the redox reaction of organic substances. There are many photocatalytic materials available, e.g., the photocatalytic materials can use g-C₃N₄ and/or FeWO₄-rGO composite material, can also use TiO₂, Bi₂WO₆, Upconversionnanoparticles@TiO₂, Ag—AgCl/α-Fe₂O₃ and the like.

The gas-liquid mixer 1 is a device for sufficiently mixing the gas and the liquid. In the mixing process, breaking the gas into small bubbles or micron and nano types of bubbles in the liquid, thereby increasing the contact area between the gas and the liquid, and effectively improving the sufficient redox reaction between Reactive Oxygen Species in the gas and materials to be treated in the liquid.

The liquid material to be detoxified and treated can be aflatoxin-contaminated cooking oil (as well as zearalenone, vomitoxin, etc.), beverage, soya milk, milk, etc., or sewage to be sterilized.

The detoxification system of the present disclosure uses the photocatalytic material and the liquid to be treated without contact to each other to carry out the detoxification treatment, which firstly avoids the secondary contamination of the liquid by the photocatalytic material, and secondly avoids the risk of quality degradation caused by the liquid being directly irradiated by the light (e.g., the light will change the acidity, the peroxide value, the unsaturated fatty acid, etc. of the peanut oil).

In a process wherein Air carrying Reactive Oxygen Species and liquid materials to be detoxified circulates in the reaction tube 4, the Reactive Oxygen Species constantly play redox reaction. However, with the continuous consumption of Reactive Oxygen Species in the air, the oxidation capacity of the gas is gradually reduced. In order to solve this problem, it is necessary to constantly filled with air carrying radiant catalytic ionized Reactive Oxygen Species, in a process of gas-liquid mixtures circulating in the reaction tube 4, and thus achieve a rapid processing effect. When the amount of Reactive Oxygen Species (ROS) in the air carrying Reactive Oxygen Species (ROS) replenished into the liquid materials is sufficient, there is no need to add new Reactive Oxygen Species into the reaction tube 4. But if the air carrying Reactive Oxygen Species (ROS) replenished into the liquid material is insufficient to treat the liquid materials to be detoxified, based on the above mentioned technical solution, it is necessary to continually replenish the air carrying radiant catalytic ionized Reactive Oxygen Species (ROS) into the gas-liquid mixture during circulating the gas-liquid mixture in the reaction tube 4.

As a specific implementation, the present disclosure provides a specific embodiment of the reaction tube 4, as shown in FIG. 4, the reaction tube 4 includes a plurality of U-shaped tubes 4-1 and a connecting tube 4-2 for connecting the U-shaped tubes 4-1; the connecting tubes 4-2 can be connected between the U-shaped tubes 4-1 by means of threaded connection, and in this way, the length of the reaction tubes can be freely designed according to the practical detoxification requirements, and the installation is convenient and fast.

During circulating the mixed materials in the reaction tube 4, the Reactive Oxygen Species in the air are continuously consumed, and the oxidation capacity of the gas gradually decreases, therefore, in order to improve the detoxification efficiency, it is necessary to maintain the content of Reactive Oxygen Species in the mixed materials. And for this purpose, it is necessary to continuously replenish new air carrying radiant catalytic ionized Reactive Oxygen Species. As a specific implementation, as shown in FIG. 4, the present disclosure designs a distal end of a straight tube section of the U-shaped tubes 4-1 as an opening structure that can be opened or closed at any time, so that when it is necessary to replenish the Reactive Oxygen Species gas, the opening is opened, and an air supplement tube 4-3 is inserted into the straight tube section from the opening, and the air carrying radiant catalytic ionized Reactive Oxygen Species are replenished into the U-shaped tubes 4-1 by the air supplement tube 4-3. The air supplement tube 4-3 is detachably connected to a distal end of a straight tube section of U-shaped tubes 4-1. When there is no need to replenish air, the distal end of the straight tube section of the U-shaped tube 4-1 can be blocked with a plug (not shown in the drawings yet). A plurality of air supplement holes 4-31 is disposed on the wall of the air supplement tube 4-3.

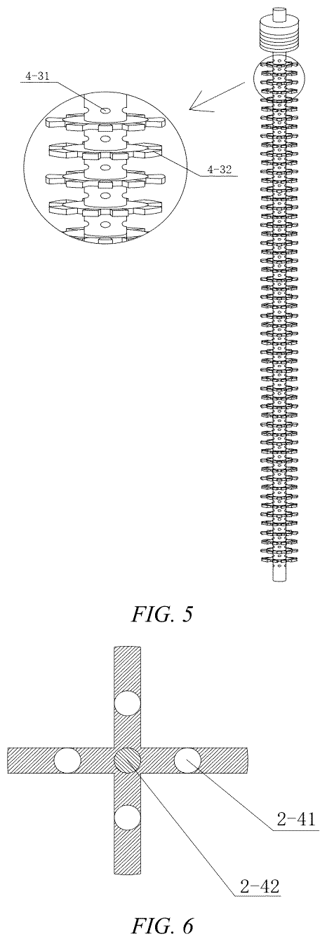

In order to improve the mixing degree between the newly replenished gas and the liquid, as shown in FIG. 5, a plurality of fins 4-32 are disposed on the air supplement tube 4-3. The provision of a plurality of fins 4-32 not only can break the newly replenished gas into small bubbles, but also at the same time, can effectively improve the mixing degree between the gas and the liquid.

Regarding to the detoxification system of the present disclosure, the amount of Reactive Oxygen Species carried in the air is crucial to the detoxification effect, in order to improve the concentration of Reactive Oxygen Species carried in the air, the present disclosure provides a specific embodiment. As shown in FIG. 2, the air carrying radiant catalytic ionized Reactive Oxygen Species are provided by an radiant catalytic ionization chamber 2; the radiant catalytic ionization chamber 2 includes a chamber body 2-1, a plurality of mesh panels 2-2 disposed in the chamber body 2-1, and a lamp tube 2-3 for providing a light source for irradiation; the mesh panels 2-2 are attached with photocatalytic material for generating Reactive Oxygen Species upon light irradiation.

A plurality of mesh panels 2-2 coated with photocatalytic materials disposed in the radiant catalytic ionization chamber 2 of the present disclosure could stimulate more Reactive Oxygen Species under irradiation of the lamp tube 2-3; the chamber body 2-1 is open at both ends, with an air inlet at one end and an air outlet at the other end, and the Reactive Oxygen Species are driven by a blower (which is not shown in the drawings yet) to enter from the chamber 2 into a gas-liquid mixer 1, so as to be mixed with the liquid material to be treated. Specifically, the mesh panels 2-2 may use a mesh panel of a light-transmitting material; and a photocatalytic material is attached to the surface of the mesh panels 2-2. Compared with the existing catalytic ionization device, the radiant catalytic ionization chamber 2 of the present disclosure uses the mesh panels 2-2 coated with the photocatalytic materials, instead of setting the photocatalytic material on a flat plate. Therefore, the present disclosure not only increases the photocatalytic material content per unit volume, but also increases the area of light illumination at the same time due to the photocatalytic material being uniformly attached to the surface of the mesh panels. At the same time, the mesh structure of the mesh panels 2-2 is more loaded with the photocatalytic material, under a premise that it meets the need of gas circulation and facilitates the circulating air to pass through mesh panels and to take away the Reactive Oxygen Species generated by the irradiation of the photocatalytic material on the mesh panel, so as to provide the system with sufficient Reactive Oxygen Species. As a specific embodiment, the mesh panels 2-2 use a light transmissive acrylic, PC or PVC rigid sheet covered with air permeable holes, and photocatalytic materials are attached to the mesh panels 2-2.

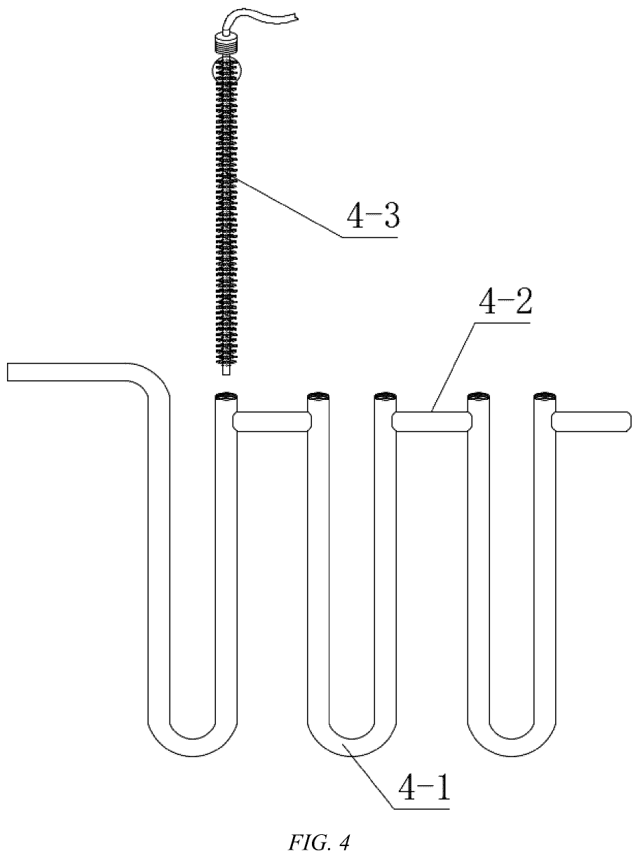

As a specific application solution, as shown in FIG. 2, in the chamber body 2-1, a fixing frame 2-4 is further provided for fixing the mesh panels 2-2 and the lamp tube 2-3; on the fixing frame 2-4, fixing holes 2-41 are provided for fixing the lamp tube 2-3, and a fixing rod 2-42 is provided for fixing the mesh panels 2-2. And as shown in FIG. 3, on the mesh panels 2-2, a mounting hole 2-21 is provided for the lamp tube 2-3 to pass through, and a fixing hole 2-22 is provided for the fixing rods 2-42 to pass through. In this way, it is possible to assemble the mesh panels 2-2, the lamp tube 2-3 and the fixing frame 2-4 outside the chamber body 2-1 in advance, to insert them into the chamber body 2-1 after assembly, and to fix the fixing frame 2-4 inside the chamber body 2-1 (the four legs of the fixing frame 2-4 are fixed to the inner wall of the chamber body 2-1). Only one embodiment of the position and number of a mounting hole 2-21 and a fixing hole 2-22 is given in FIG. 3. And when used, the position and number of the mounting hole 2-21 and the fixing hole 2-22 can be freely set according to requirements.

The structure of the fixing frame 2-4 is shown in FIG. 6, because the chamber body 2-1 is ventilated therein when in use, in order to increase the stability of the mesh panels 2-2 and the lamp tube 2-3, as shown in FIG. 2, a fixing frame is added onto the other side of the plurality of mesh panels (the left side in the FIG. 2), and the middle of this fixing frame consists of a round hole instead of the fixing rod, the round hole being used for insertion of the fixing rod 2-42, which will not be described herein.

The specific type of the lamp tube 2-3 is not particularly limited, as long as it is capable of irradiating the catalytic semiconductor material to produce Reactive Oxygen Species, such as ordinary fluorescent lamp tubes or lamp tubes that produce special wavelengths of light, such as in embodiment 3 wherein a 300 W xenon lamp and an optical cut-off filter are used.

When in use, the lamp tube 2-3 in the radiant catalytic ionization chamber 2 are turned on for irradiation catalysis, and a pump is used to pump the material to be treated in the material-liquid tank 3 into the gas-liquid mixer 1. At the same time, a blower is used to replenish the Reactive Oxygen Species produced by the radiant catalytic ionization chamber 2 into the gas-liquid mixer 1 to mix it with the liquid materials, and the mixed materials are pumped by the pump into the reaction tube 4, so as to be detoxified. Inside the reaction tube 4, the oxidising capacity of the gas gradually decreases, so in order to improve the detoxification efficiency, the air supplement tube 4-3 is used to replenish gas to the reaction tube 4.

### Embodiment 2

Based on the detoxification system in embodiment 1, the present embodiment 2 provides a photocatalytic material-FeWO₄-rGO composite material that can be used in the detoxification system, the FeWO₄-rGO composite material being prepared by the following methods:

### (1) Preparation of FeWO₄

Firstly dissolving 5 mmol FeCl₃·6H₂O and 5 mmol Na₂WO₄·2H₂O respectively into 25 ml distilled water. Then, adding 0.5 mmol ascorbic acid into FeCl₃ solution under continuous stirring for complete dissolution. Next, slowly adding Na₂WO₄ solution into the above mixture. After further stirring for 30 min, adding the mixture into 100 mL autoclave (the autoclave in this embodiment 2 was only used as a reaction vessel), and maintaining at 180° C. for 12 h. Then naturally cooling down the autoclave to room temperature. Collecting a resulting precipitate by centrifugation, and washing with distilled water and anhydrous ethanol several times, and drying in air at 80° C. for 6 h.

### (2) Preparation of FeWO₄-rGO Composite Material

Firstly adding 2 g FeWO₄ into 300 mL ethanol, and ultrasonic treating a mixture of the 2 g FeWO₄ and 300 mL ethanol (ultrasonic condition 300 W) for 30 min. After adding 2 m LAPTES into FeWO₄ suspension, heating the mixture at 70° C. for 4 h. Subsequently collecting the powder, washing with ethanol for several times, then drying it at 80° C. for overnight. Afterwards, adding 1 g APTES-modified FeWO₄ into 60 mL distilled water, and ultrasonic treating (ultrasonic condition 300 W) for 15 min, and then adding 0.05 g rGO. After stirring for 60 min, transferring a resulting suspension into an autoclave, and maintaining it at 180° C. for 12 h. Collecting a resulting product, washing with water, and drying at 80° C. for overnight, to obtain FeWO₄-rGO.

### Embodiment 3

In order to compare the superiority of charge modification method, snowflake-like FeWO4 prepared in step (1), FeWO₄-rGO-U (FeWO₄-rGO-U is a mixture of FeWO₄ and rGO milled and mixed directly) and FeWO₄-rGO prepared by the above method were compared and tested, respectively.

### (1) XRD Test

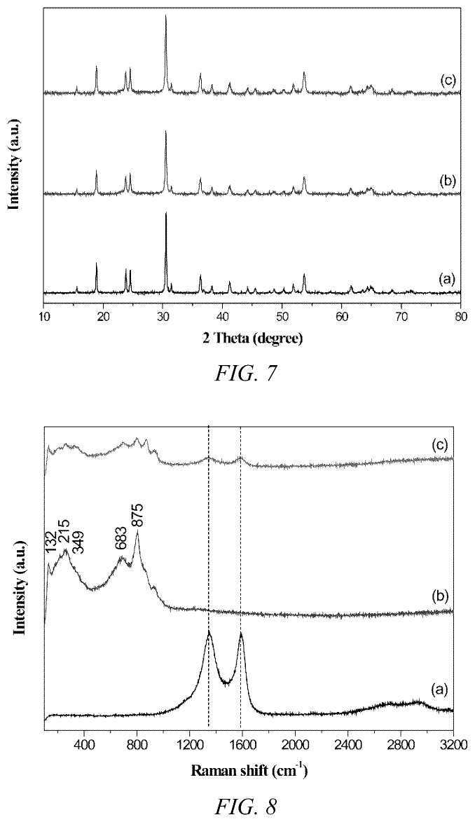

The results are shown in FIG. 7. All diffraction peaks of the product can be attributed to the standard card of FeWO₄ (JCPDS No. 46-1446). In addition, no diffraction peaks of rGO were observed.

### (2) Raman Spectroscopy Test

The results are shown in FIG. 8, and the presence of rGO in the complex was further determined by Raman analysis. As shown in FIG. 8 (a), two main peaks were observed near 1596 and 1346 cm⁻¹, which should be attributed to the G band and D band of GO. The peaks at 93, 132, 215, 179, 349, 683 and 875 cm⁻¹ correspond to the Raman spectrum of FeWO₄, as shown in (b) in FIG. 8. Regarding to the FeWO₄-rGO composite material, in addition to the peaks attributed to FeWO₄, the G band and D band of rGO are located at 1598 cm⁻¹ and 1345 cm⁻¹, respectively. This clearly indicates the presence of rGO in the prepared rGO-FeWO₄ composites.

### (3) UV-Vis Diffuse Reflection

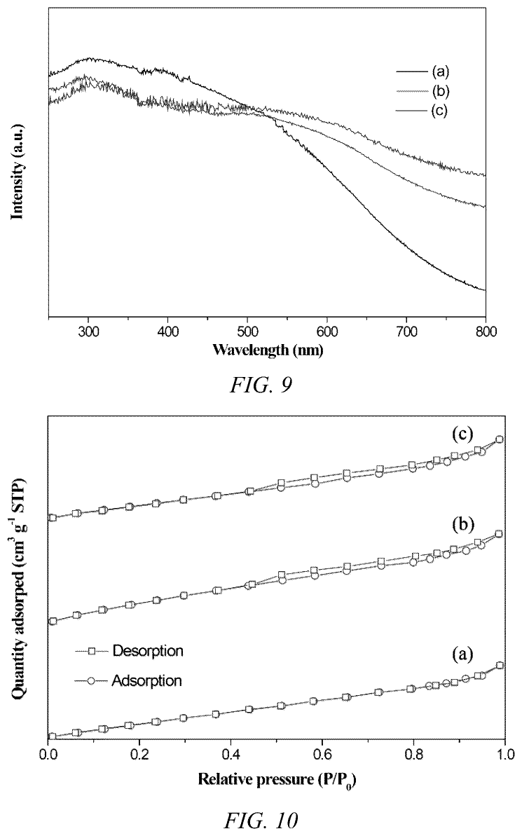

The results are shown in FIG. 9, the optical properties of the material can be characterized by UV-vis DRS spectrum; and the snowflake-like FeWO⁴ shows strong absorption in the visible region. Enhanced absorption of visible light in the region between 500 and 800 nm can be clearly observed, by adding rGO. Extension of the light absorption range enables more efficient use of the solar spectrum and enhanced photocatalytic activity. In addition, the background absorption of FeWO⁴-rGO composite material is significantly enhanced in the visible region.

### (4) N₂ Adsorption and Desorption

The N₂ adsorption-desorption isotherms of the prepared products are shown in FIG. 10. The specific surface areas of FeWO₄-rGO, FeWO₄-rGO-U and pure snowflake-like FeWO₄ were 49.18, 47.03 and 45.80 cm³g⁻¹, respectively, with no significant difference in values.

### (5) SEM Test and TEM Test

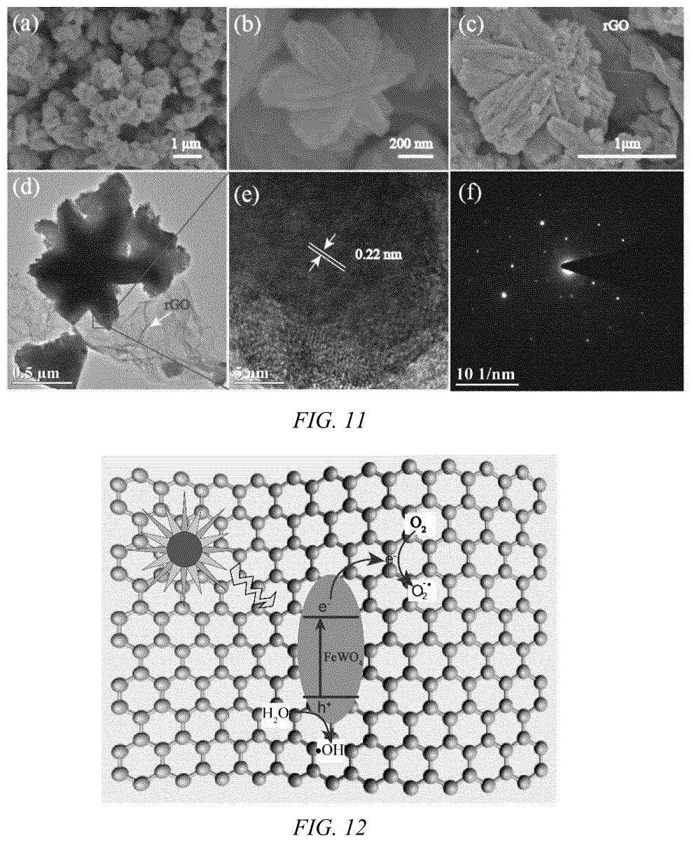

The results are shown in FIG. 11, wherein the morphology and structure of snowflake-like FeWO₄ and FeWO₄-rGO composite materials were investigated by SEM and TEM. As shown in (a) of FIG. 11, FeWO₄ consists of uniform snowflake-like microcrystals with a diameter of about 1 μm. High-magnification SEM shows that each snowflake-like FeWO₄ with approximate sixfold symmetry consists of nanorods assembled in specific directions (b in FIG. 11). As for the FeWO₄-rGO materials (c-d in FIG. 11), FeWO₄ is wrapped by rGO nanosheets, which implies the possibility of full contact and effective interfacial interactions between FeWO₄ and rGO. The TEM of the FeWO₄-rGO composite material is shown in FIG. 11, (d). The FeWO₄ assemblies are deposited on the surface of the rGO sheets. The sample shows some folds at the edges of both the rGO sheet and the sandwich sheet, providing more adsorption active sites. A well-defined lattice stripe with a spacing of 0.22 nm, which corresponds to the (020) plane of FeWO₄, is shown in (e) of FIG. 11. In addition, selected area electron diffraction (SAED) shows well-defined rings, which confirms the polycrystalline nature of the sample.

As shown in FIG. 12, under visible light irradiation, electrons in the valence band (VB) are excited into the conduction band (CB), leaving holes in the VB and generating electron-hole pairs in FeWO₄. Photogenerated electrons (e⁻) are then transferred from the FeWO₄ microstructure to the rGO sheet and further react with O₂ to produce highly reactive free radicals (O²⁻). Holes in the VB of FeWO₄ react with water to form reactive OH⁻. These free radicals are capable of attacking fungal toxins and abrogating the fungal toxins according to photocatalytic redox reactions. On the other hand, the surface charge modification provides a rational method for constructing FeWO₄-rGO nanocomposite photocatalysts with sufficient interfacial contacts according to electrostatic self-assembly, as compared to FeWO₄-GO-U composite material. All of the above factors contribute to the improvement of the separation efficiency of electron-hole pairs, which significantly enhances the photoactivity of the FeWO₄-rGO composite material.

### Morphological Changes of the Generated FeWO₄ Under Different Temperature Conditions

Firstly dissolving 5 mmol FeCl₃·6H₂O and 5 mmol Na₂WO₄·2H₂O respectively into 25 ml distilled water. Then, adding 0.5 mmol ascorbic acid into the FeCl₃ solution under continuous stirring for complete dissolution. Next, slowly adding Na₂WO₄ solution into the above mixture. After further stirring for 30 min, the mixture was added into 100 mL autoclave, maintaining it at a certain temperature for 12 h. Then naturally cooling down the autoclave to room temperature. Collecting a resulting precipitate by centrifugation, and washing several times with distilled water and anhydrous ethanol, and drying it in air at 80° C. for 6 h.

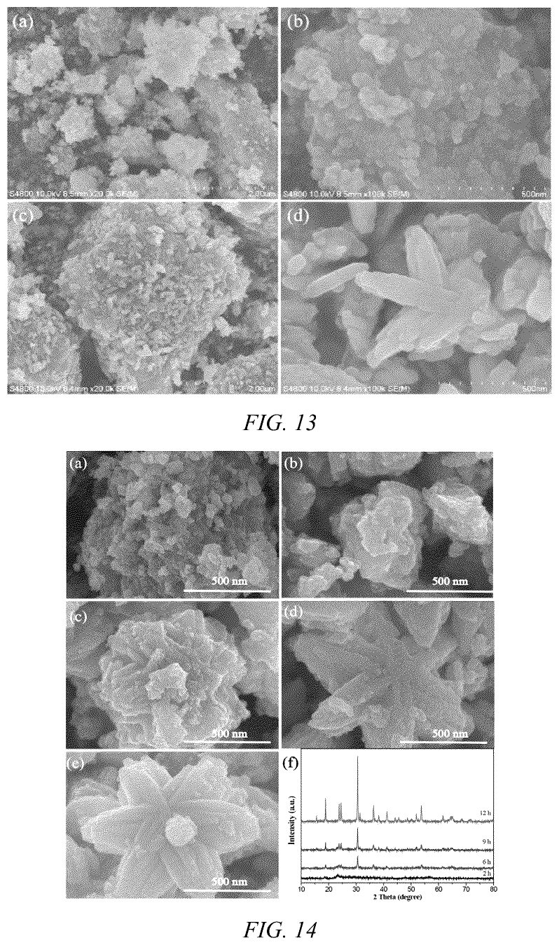

The results are shown in FIG. 13. FIG. 13 shows the effect of temperature on the morphology of FeWO₄ samples. Irregular shapes were observed at 120° C. ((a) in FIG. 13). Upon increasing the temperature to 150° C., the average size of the products was smaller, although the morphology of the products was similar to that of the 120° C. hydrothermal-treated samples. When the temperature was increased to 180° C. ((a) and (b) in FIG. 11), the prepared samples consisted of snowflakes with perfect homogeneity. When the temperature was increased to 200° C., the surface of the nanorods became smoother, although the samples maintained a snowflake-like structure ((c) and (d) in FIG. 13). This result also suggests that the final morphology depends on the formation temperature of FeWO₄ crystals.

### Morphological Changes of the Generated FeWO₄ Under Different Time Conditions

Firstly, dissolving 5 mmol FeCl₃·6H₂O and 5 mmol Na₂WO₄·2H₂O respectively into 25 ml distilled water. Then, adding 0.5 mmol ascorbic acid into the FeCl3 solution under continuous stirring for complete dissolution. Next, slowly adding Na₂WO₄ solution into the above mixture. After further stirring for 30 min, adding the mixture into a 100 mL autoclave and maintaining it at 180° C. for a certain period of time. Then naturally cooling the autoclave to room temperature. Collecting a resulting precipitate by centrifugation, and washing it several times with distilled water and anhydrous ethanol, and drying it in air at 80° C. for 6 h.

In order to understand the growth mechanism of snowflake-like FeWO₄, SEM of the material prepared at different hydrothermal times is shown in FIG. 14. When the reaction time was lower than 2 h, the products consisted of large ovoid particles ((a) and (b) in FIG. 14). The image of the sample reacted for 6 h is shown in (c) in FIG. 14, indicating the formation of plate-like aggregates. These aggregates were formed from many nanoparticles and nanorods. As the process continues, snowflake-like FeWO₄ structures appear after 9 h, showing hexagonal symmetry ((d) in FIG. 14). It should be noted that the nanorods extend outwards from the center of the aggregates in a self-assembled form. As the reaction continued, the irregular particles disappeared and longer nanorods were formed, suggesting that the longer nanorods grew at the expense of smaller particles. After 12 h, the nanoparticles on the snowflake surface of the FeWO₄ grew along its axis, to form nanorods ((e) in FIG. 14). These images clearly show the shape evolution process of the obtained product from aggregates built of nanoparticles to snowflake-like structures. XRD patterns of FeWO₄ samples prepared at different hydrothermal times are shown in (f) in FIG. 14. The crystalline phase is enhanced with increasing hydrothermal time. All XRD patterns can be easily identified as FeWO₄.

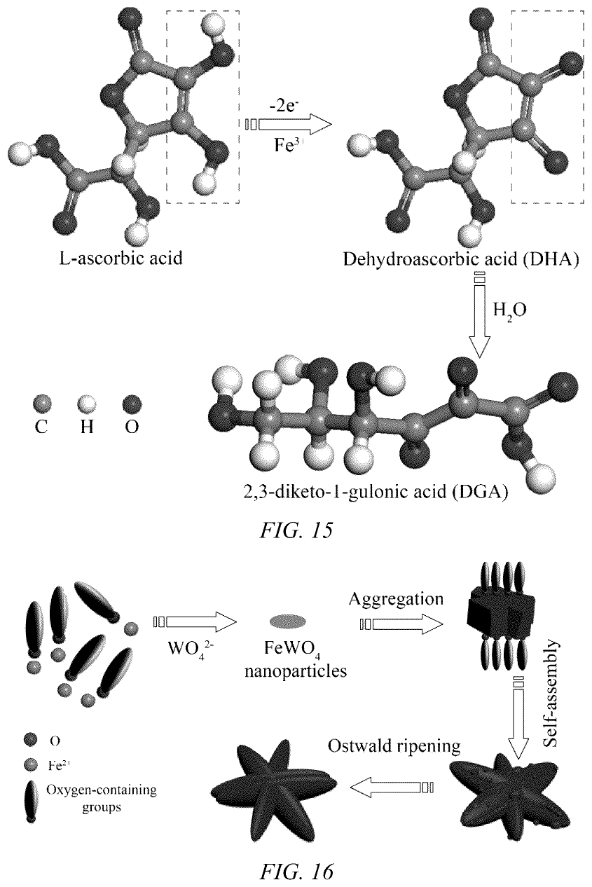

Based on the above analysis, the shape evolution process of the snowflake-like microstructures is shown in FIG. 15 and FIG. 16. During this process, oxygen-containing groups (e.g., −OH and C═O) in DHA and DGA strongly tend to coordinate with Fe²⁺ cations and form complexes. As a result, the concentration of free Fe²⁺ cations decreases. The slow formation rate of FeWO₄ will lead to a separation of a nucleation step and a growth step. Then WO₄²⁻ reacts with the complexes to form FeWO₄ nuclei. DHA and DGA in the reaction system are absorbed by the surface of the FeWO₄ nuclei. At the same time, the FeWO₄ nuclei tend to aggregate, due to the decrease in the surface energy of the nuclei and the hydrogen interaction between DHA and DGA. In addition, due to the high intrinsic anisotropic nature of FeWO₄, the nanoparticles preferred to grow into nanorods by directional attachment. With time prolongation of hydrothermal treatment, snowflake-like microstructures were formed. We can conclude that the formation of graded snowflake-like microstructures is a result of ascorbic acid-induced reduction, formation and aggregation of nanoparticles followed by growth of nanorods and their subsequent self-assembly. According to the reported literature, after modification by APTES, FeWO₄ has amine functional groups. Under the effect of electrostatic attraction, FeWO4 was anchored to the surface of negatively charged rGO nanosheets.

### Effect of Different Amounts of Ascorbic Acid on Product Morphology

Firstly dissolving 5 mmol FeCl₃·6H₂O and 5 mmol Na₂WO₄·2H₂O respectively into 25 ml distilled water. Then, adding a certain amount of ascorbic acid into FeCl₃ solution under continuous stirring for complete dissolution. Next, slowly adding Na₂WO₄ solution into the above mixture. After further stirring for 30 min, adding the mixture into a 100 mL autoclave, and maintaining it at 180° C. for 12 h. Then naturally cooling down the autoclave to room temperature. Collecting a resulting precipitate by centrifugation, and washing with distilled water and anhydrous ethanol several times, and drying in air at 80° C. for 6 h.

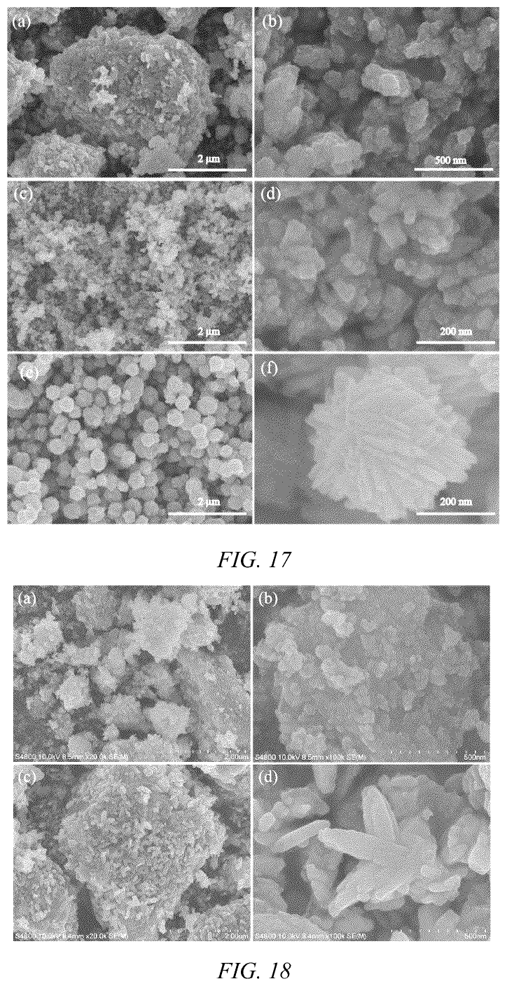

The effect of different amounts of ascorbic acid on the morphology of the product was studied and the studied results are shown in FIG. 17. FIG. 17 shows SEM images of the samples prepared in a case where different amounts of ascorbic acid (0.2 to 1 mmol) are present in the reaction system. When 0.2 mmol of ascorbic acid was added, disordered tiny particles were the main products ((a) (b) in FIG. 17). After increasing the amount of ascorbic acid (0.3 mmol), some particle aggregates were formed ((c) (d) in FIG. 17). Further increasing the amount of ascorbic acid to 0.5 mmol, the prepared samples consisted of snowflakes with perfect homogeneity and monodispersity ((a) (b) in FIG. 11). When the amount of ascorbic acid was up to 1 mmol, the product showed a spherical shape and consisted of nanorods ((e) (f) in FIG. 17). Thus, the successful preparation of novel FeWO4 nanostructures suggests that ascorbic acid can be used in this system not only as a reducing agent but also as a structure-directing agent to synthesize FeWO₄ products.

### Effect of Different Raw Materials on FeWO₄

In case where step (1) of embodiment 2 does not change, respectively, A only replacing FeCl₃·6H₂O with equimolar Fe(NO₃)₃·9H₂O, and Bonly adding 0.1 mmol of cetyltrimethylammonium bromide when adding ascorbic acid.

The results are shown in FIG. 18, wherein the morphology and size of the FeWO₄ products depend on the iron precursor salt used. The anion of the iron precursor salt determines the final structure of FeWO₄. Under similar conditions, a uniform snowflake-like structure assembled from nanorods could not be obtained by only using Fe (NO₃)₃ instead of FeCl₃. The corresponding SEM is shown in FIG. 18 (a) (b). In addition to the iron precursor salt, we found that the presence of CTAB also affects the morphology of the final product, as shown in FIG. 18 (c) (d). In case that CTAB exists, the final product can also consist of nanorods, but the snowflake-like organized structure would not exist at all. Thus, CTAB can further provide additional control over the morphology of the product.

Aflatoxin (AFB1) degradation experiments were performed as following steps of:

Coating 500 mg of flower-like FeWO₄-rGO composite material prepared in embodiment 2 on a glass substrate, and then adding it into 100 mL of corn oil (concentration 16.8 ppb) containing AFB1 with 500 rpm magnetic stirring. And then placing it in the dark and stirring it for 30 min, so as to achieve adsorption-desorption equilibrium. Using a 300 W xenon lamp to irradiate the suspension. Collecting 5 mL of corn oil at 0 min, 15 min, 30 min, 45 min, 60 min, and 75 min of irradiation time, respectively. The concentration of AFB1 in peanut oil was tested by liquid chromatography, and the results are shown in Table 1. The control groups were snowflake-like FeWO₄ and FeWO₄-rGO-U prepared as comparison examples.

TABLE 1

| Light time (min) | 0 | 15 | 30 | 45 | 60 | 75 |
|---|---|---|---|---|---|---|
| AFB₁concentration (ppb) in | 16.8 | 15.3 | 14.2 | 13.6 | 12.8 | 13.8 |
| snowflake-like FeWO4group | | | | | | |
| AFB₁concentration (ppb) in | 16.8 | 14.6 | 11.2 | 9.6 | 8.7 | 6.2 |
| snowflake-like FeWO4-rGO- | | | | | | |
| Ugroup | | | | | | |
| AFB₁concentration (ppb) in | 16.8 | 12.1 | 9.6 | 4.1 | 3.6 | 2.5 |
| snowflake-like FeWO4- | | | | | | |
| rGOgroup | | | | | | |

It can be seen from Table 1, in comparison with snowflake-like FeWO₄ and FeWO₄-rGO-U, flower-like FeWO₄-rGO has excellent photocatalytic degradation performance for corn oil AFB₁.

### Application Example 2

Escherichia coli bacterial fluids were mixed with the prepared materials, and irradiated with an optical cut-off filter (λ>420 nm) using a 300 W xenon lamp for antimicrobial performance evaluation experiments. The bacterial suspensions with different irradiation time intervals (0 min, 5 min, 10 min, 15 min, 20 min, 25 min) were taken and coated on LB agar plates, and incubated for 4 h at 37° C. in a thermostat to observe the growth of the colonies. At the same time, the bacterial fluids were diluted, and plate counting was performed, so as to determine the number of surviving bacteria and calculate the bactericidal efficiency.

$$Bactericidal efficiency ( \% ) = ( {N}_{0} - {N}_{t} ) / {N}_{0} \times 100 \%$$

Note: N₀ and Nₜ in the formula are the number of colonies counted in the control group and the sample plate respectively.

The experiment results are shown in Table 2.

TABLE 2

| Light time (min) | 0 | 5 | 10 | 15 | 20 | 25 |
|---|---|---|---|---|---|---|
| Bactericidal efficiency of | 1 | 0.91 | 0.83 | 0.79 | 0.75 | 0.71 |
| snowflake-like FeWO₄ group | | | | | | |
| Bactericidal efficiency of | 1 | 0.84 | 0.71 | 0.63 | 0.59 | 0.52 |
| snowflake-like FeWO₄-rGO- | | | | | | |
| U group | | | | | | |
| Bactericidal efficiency of | 1 | 0.65 | 0.42 | 0.36 | 0.21 | 0.08 |
| snowflake-like FeWO₄-rGO | | | | | | |
| group | | | | | | |

As can be seen from Table 2, compared with snowflake-like FeWO₄ and FeWO₄-rGO-U, snowflake-like FeWO₄-rGO prepared in the present disclosure has excellent photocatalytic killing performance against Escherichia coli.

The foregoing is only a preferred embodiment of the present disclosure, and is not intended to be a limitation of the present disclosure in any other form, and any skilled person familiar with the art may use the technical content disclosed above to change or reformat into equivalent embodiments of equivalent changes. However, any simple modification, equivalent change or transformation of the above embodiments based on the technical substance of the present disclosure without departing from the content of the technical solutions of the present disclosure still belongs to the protection scope of the technical solutions of the present disclosure.

What is claimed is:

1. A radiant catalytic ionization detoxification system, comprising a gas-liquid mixer (1) configured to gas-liquid mix air carrying radiant catalytic ionized Reactive Oxygen Species with liquid material to be detoxified, and a reaction tube (4) configured to circulate the mixed gas-liquid mixture;
wherein the reaction tube (1) comprises a plurality of U-shaped tubes (4-1), and a connecting tube (4-2) configured to connect the U-shaped tubes (4-1); an air supplement tube (4-3) is detachably connected at a distal end of a straight tube section of the U-shaped tubes (4-1), the air supplement tube (4-3) being configured to introduce air carrying radiant catalytic ionized Reactive Oxygen Species into the U-shaped tubes (4-1); a plurality of air supplement holes (4-31) are disposed on a wall of the air supplement tube (4-3);
a plurality of fins (4-32) are disposed on the air supplement tube (4-3);
the air carrying radiant catalytic ionized Reactive Oxygen Species are provided by a radiant catalytic ionization chamber (2); the radiant catalytic ionization chamber (2) comprises a chamber body (2-1), a plurality of mesh panels (2-2) disposed in the chamber body (2-1) and a lamp tube (2-3) configured to provide a radiant light source; a photocatalytic material which produces Reactive Oxygen Species upon light irradiation is coated on the mesh panels (2-2);
a fixing frame (2-4) is further disposed in the chamber body (2-1), the fixing frame (2-4) being configured to fix the mesh panels (2-2) and the lamp tube (2-3); a fixing hole (2-41) configured to fix the lamp tube (2-3), and a fixing rod (2-42) configured to fix the mesh panels (2-2) are disposed on the fixing frame (2-4); a mounting hole (2-21) configured to be passed through by the lamp tube (2-3), and a fixing hole (2-22) configured to be passed through by the fixing rod (2-42) are disposed on the mesh panels (2-2); and
the photocatalytic material is FeWO₄-rGO composite material.

2. The radiant catalytic ionization detoxification system according to claim 1, wherein the FeWO₄-rGO composite material is prepared by the following method:
adding FeWO₄ into ethanol and ultrasonically treating a mixture of FeWO₄ and ethanol; after adding APTES into FeWO₄ suspension, heating the mixture at 70° C. for 4 h; subsequently collecting the powder, washing with ethanol, and drying; and then, adding APTES-modified FeWO₄ into distilled water, and ultrasonically treating a mixture of APTES-modified FeWO₄ and distilled water, and then adding rGO; after stirring for 60 min, maintaining resultant suspension at 180° C. for 12 h; finally collecting a resulting product, washing with water, and drying at 80° C., thereby obtaining FeWO₄-rGO.

3. The radiant catalytic ionization detoxification system according to claim 2, wherein the FeWO₄ is prepared by the following method:
firstly dissolving FeCl₃·6H₂O and Na₂WO₄·2H₂O respectively into distilled water; then, adding ascorbic acid into FeCl₃ solution during continuous stirring for complete dissolution; next, slowly adding Na₂WO₄ solution into the above mixture; after further stirring, maintaining the mixture at a high temperature for a certain period of time; then naturally cooling down to room temperature; collecting a resulting precipitate by centrifugation, and drying after washing.

4. An application of the radiant catalytic ionization detoxification system according to claim 1 in water sterilization or aflatoxin removal in edible oils.

5. A radiant catalytic ionization detoxification method, applied to the system according to claim 1, comprises a step of generating Reactive Oxygen Species according to radiant catalytic ionization, a step of gas-liquid mixing air carrying radiant catalytic ionized Reactive Oxygen Species with liquid materials to be detoxified, and a step of detoxifying bacteria or aflatoxin in liquid materials by Reactive Oxygen Species.
